(19) United States
(12) Patent Application Publication
(10) Pub. No.: US 2017/0071582 A1
(43) Pub. Date: Mar. 16, 2017

(54) FLUID SAMPLE COLLECTION AND PRESERVATION SYSTEM

(71) Applicant: OASIS DIAGNOSTICS CORPORATION, VANCOUVER, WA (US)

(72) Inventors: ROBERT L. BUCK, FAIRVIEW, OR (US); JASON GIDDINGS, FOREST GROVE, OR (US); PAUL D. SLOWEY, VANCOUVER, WA (US)

(73) Assignee: OASIS DIAGNOSTICS CORPORATION, VANCOUVER, WA (US)

(21) Appl. No.: 14/804,309

(22) Filed: Jul. 20, 2015

Related U.S. Application Data
(60) Provisional application No. 62/026,542, filed on Jul. 18, 2014.

(51) Int. Cl. A61B 10/00

(52) U.S. Cl. CPC A61B 10/0051; A61B 10/0096

(57) ABSTRACT

A sample collection system includes a sample collector having a handle with a collection strip retainer and male threaded portion disposed around the retainer, a collection strip extending from the retainer, wherein the collection strip has a pre-determined maximum load for the sample fluid and a sample volume adequacy indicator; a transport tube including a coupling to removably receive a transport tube cap and a female threaded portion to removably receive the sample collector male threaded portion, the transport tube including visually discernable graduated scale and being a translucent or transparent; and, a predetermined volume of buffer solution disposed within the transport tube cylinder. A method for collecting a fluid sample of adequate volume and known dilution factor is provided utilizing the sample collection system.

### FIELD OF THE INVENTION

The present invention relates to whole saliva sample collection and preservation. More particularly, the present invention relates to oral fluid collection systems and methods intended for the controlled and standardized collection and transportation of oral fluid specimens for the purpose of subsequent testing for various drug molecules, metabolites, steroid hormones, and other molecules, useful for drug and drug metabolite testing, including but not limited to saliva and urine, and for field test kits for chemical exposure and drug use.

### BACKGROUND

Saliva-based testing for drugs of interest and chemical exposures is becoming more desirable and widespread versus blood testing for several reasons, including that safety protocols for handling saliva are less cumbersome than for blood products, and that obtaining the tests is less intrusive and therefore easier to obtain permission. However, accuracy is an immense concern, especially in the context of testing for illegal drugs where false positives and false negatives can have major negative consequences.

Among the significant difficulties in using saliva for testing for levels of drug presence—as opposed to simply testing for exposure—are the related issues of adequate sample volume and accurately determining the dilution level in the preserved sample. Even where reliable sample volume adequacy indicators (SVAI) are provided, the actual volume of saliva collected can vary significantly because the SVAI indicates only that some minimum sample volume is reached, usually based on having sufficient sample to obtain a minimum detection threshold, but does not prevent over-collection, nor is even this minimum volume indication very precise. Many drug tests require accurate determination of the dilution level of the saliva sample in order to reliably indicate the actual level of the drug or metabolite in the subject's system rather than just determining it is above a minimum threshold, so merely including an SVAI does not resolve this problem. In other words, the SVAI indicates adequacy for detection, but does not reliably indicate actual volume of sample collected.

Generally, test strips are usable, but for go/no-go testing, or for complicated lab analysis driven by the fact that the actual volume of whole saliva—and hence the actual dilution ration of the buffered stored sample—is not known, making tests to determine actual levels of chemicals or biochemical markers of interest difficult and unreliable, This unreliability is especially true where samples are collected in field conditions, or for large scale screening/research programs.

The only reliable method existing is to collect a volume of saliva by spit collection or vacuuming. Vacuum collection requires relatively advanced facilities and is expensive to maintain. Spit collection—i.e. where the patient expresses saliva directly into a volume collector such as a spit cup—is very time consuming, and prone to contamination in field conditions. It is also somewhat off-putting to many patients, who may consider it undignified. For patients with low saliva production, which can be caused by underlying medical conditions, treatments such as chemotherapy, stress, or drug/chemical exposure, collecting samples by direct expression can be very difficult.

Despite the availability of a number of oral fluid collection devices, there are few offering standardized collection of oral fluids for drug and chemical metabolite testing with a means of confirmation of sample sufficiency. Applicant's invention incorporates a novel sample volume adequacy indicator built into the device handle and provides a suitable quantity of oral fluids and whole saliva for a variety of drug and chemical testing applications.

The collection of alternative specimens such as urine, hair, sweat, tears, saliva and others is growing in importance as an alternative to blood sampling. A number of devices exist that allow for the collection of various forms of oral fluid or saliva and each has specific application in the growing market for salivary diagnostic testing.

Despite the availability of a number of oral fluid collection devices, there are few offering standardized collection of oral fluids for drug and drug metabolite testing with a means of confirmation of sample sufficiency. The collector system incorporates a novel Sample Volume Adequacy Indicator (SVAI) built into the device handle and provides a suitable quantity of oral fluids (whole saliva) for a variety of drug testing applications. In addition the device provides a critical means of determining sample dilution when insufficient sample is collected.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows a side view of a collection system with collector inserted, and a collection system with collector separated.

FIG. 2 shows a top view of the items shown in FIG. 1.

FIG. 3 shows a bottom view of the items shown in FIG. 1.

FIG. 4 shows a perspective view of the items shown in FIG. 1.

FIG. 5 shows an illustrated sample collection procedure.

FIG. 6 shows a side view of an SVAI portion.

FIG. 7 shows a view of a collector and transport tube of a first embodiment, with greater detail of the calibrated scale.

FIG. 8 shows a side view of a system, in preparation for use.

FIG. 9 shows a close up of an SVAI portion, in preparation for use.

FIG. 10 shows a sample collector being inserted into a subject's mouth for collection or oral fluid sample.

FIG. 11 shows pre-sample and post-sample view of a collector.

FIG. 12 demonstrates the removal of the storage cap, insertion of a post-collection sample collector into the transport tube, and sealing of the tube for transfer of the sample.

FIG. 13 shows a post-collection transport tube with sample collector inserted being agitated.

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

The fluid collection system and methods described herein provide for the standardized collection of bodily fluids. The described embodiment is described by way of example in terms of collecting whole saliva from the side of the tongue in the mouth, or other bodily fluids from appropriate sample locations or reservoir. The described embodiment is optimized for direct saliva collection from the mouth of a subject, and includes a collection strip comprised of an absorbent pad material, which is placed in the pool of saliva under the tongue and adjacent to the teeth to collect the sample. After 1-2 minutes, a sample volume adequacy indicator changes appearance signifying sufficient saliva has been collected for subsequent analysis. The collection strip is then transferred to a secondary tube for transportation to a laboratory. Upon receipt at the laboratory the sample may be removed by vortexing or centrifugation if it has received insufficient inversion during transit and is then ready for drug analysis by various methods.

Under field conditions, it is not always possible to obtain an adequate quantity of saliva, especially in cases where the subject/donor is suffering from dry mouth. In these instances the quantity of saliva collected using the system can be accurately calculated from the amount of liquid displaced in the transport tube when the collection strip is introduced into the transport buffer, using the conversion equation provided.

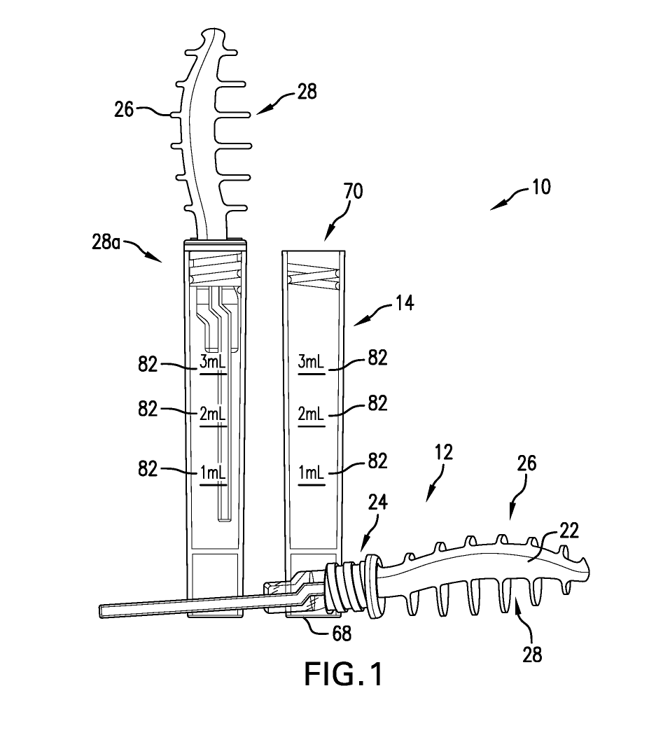

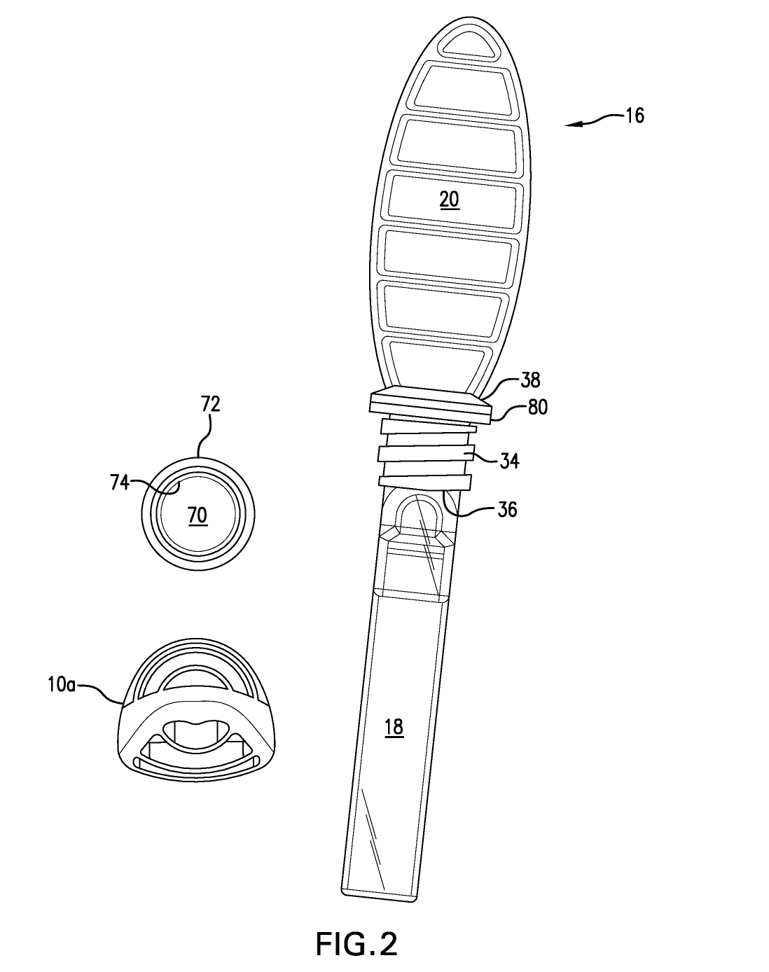

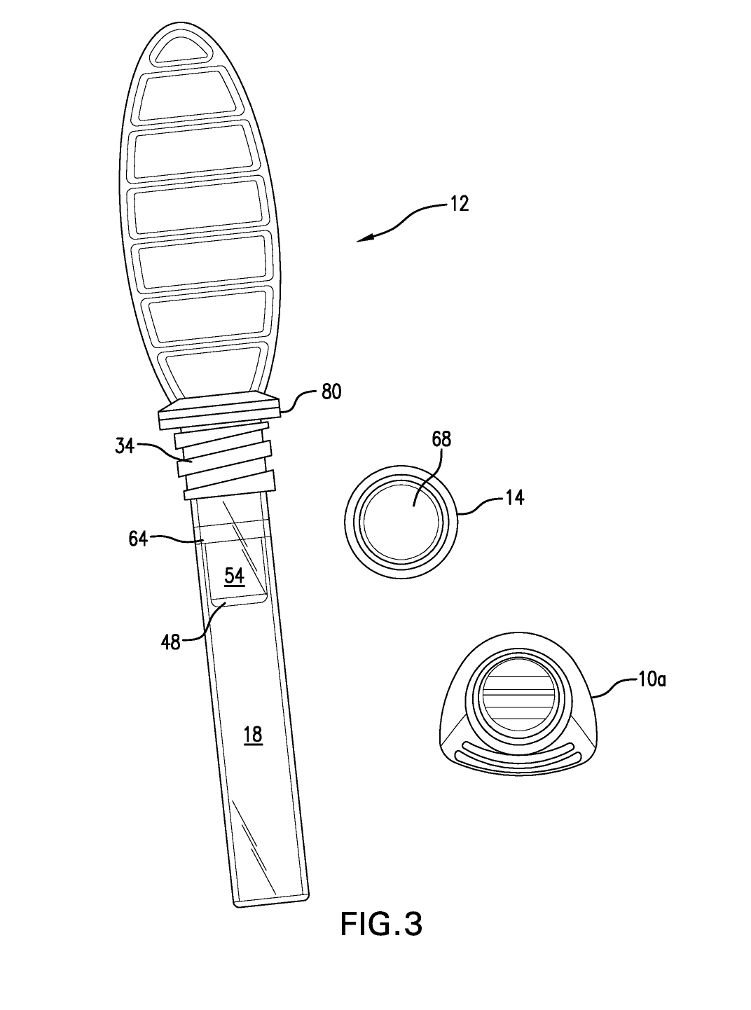

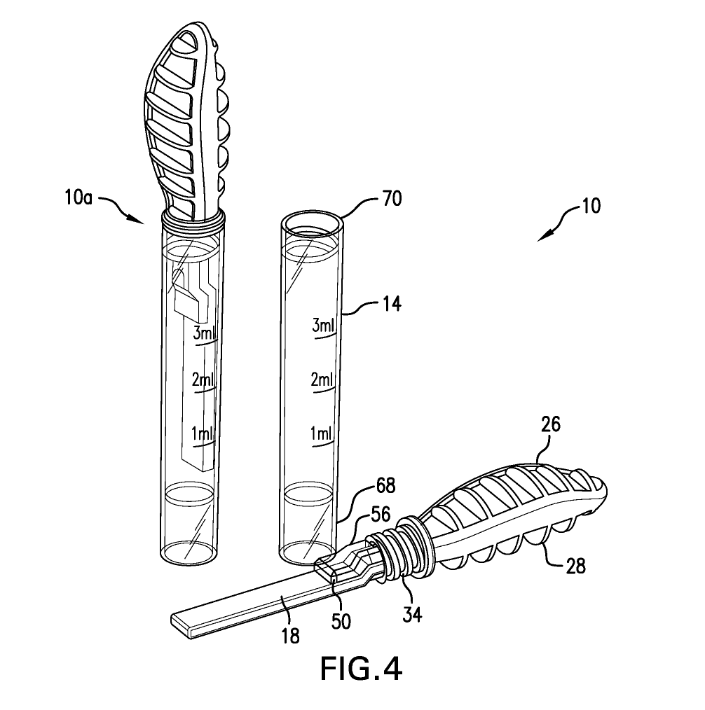

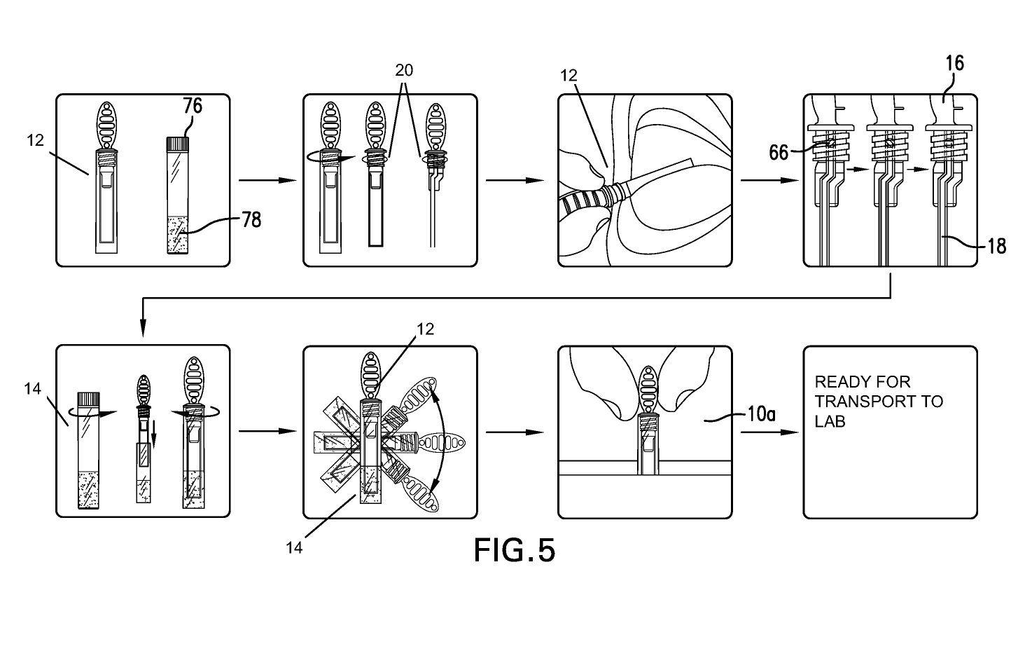

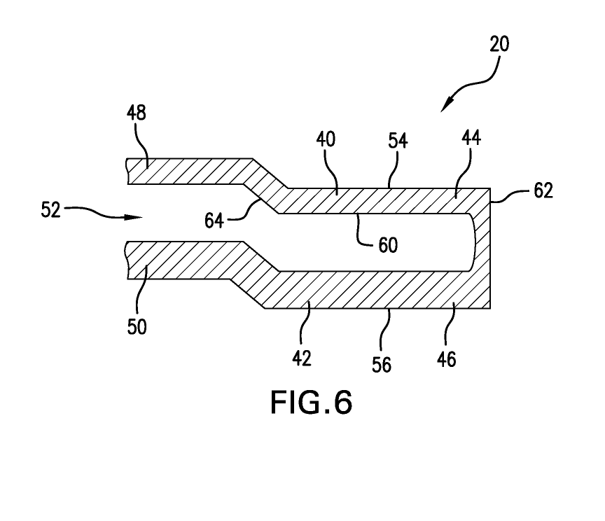

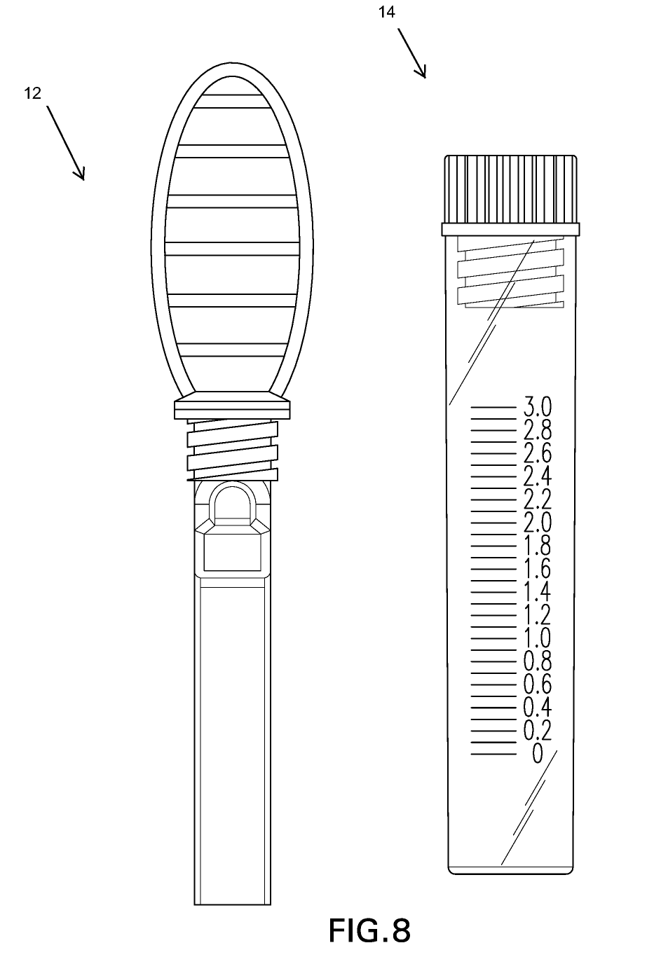

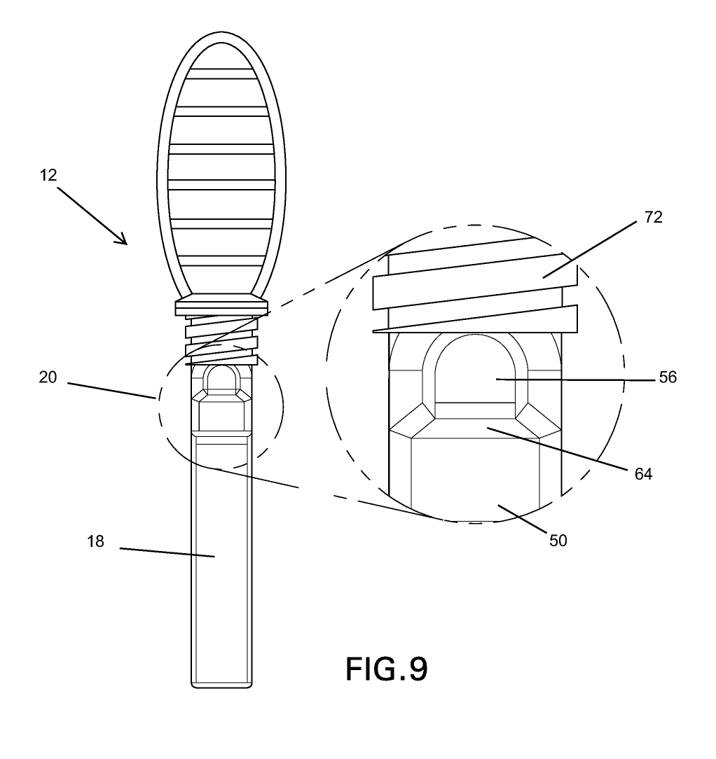

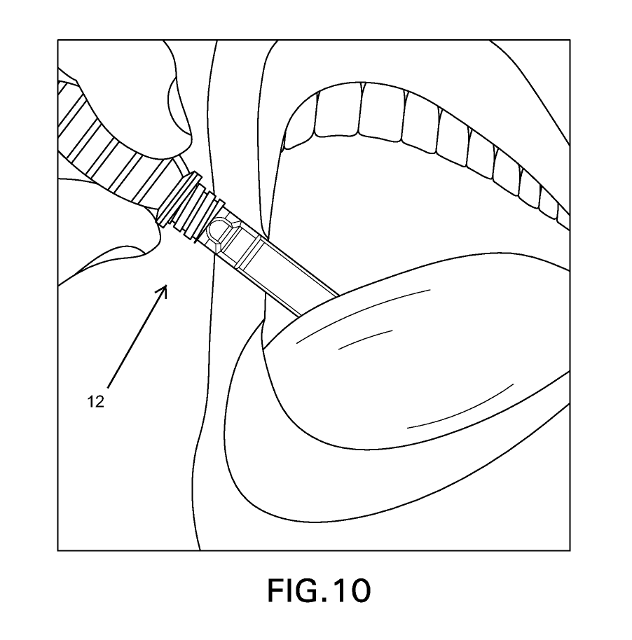

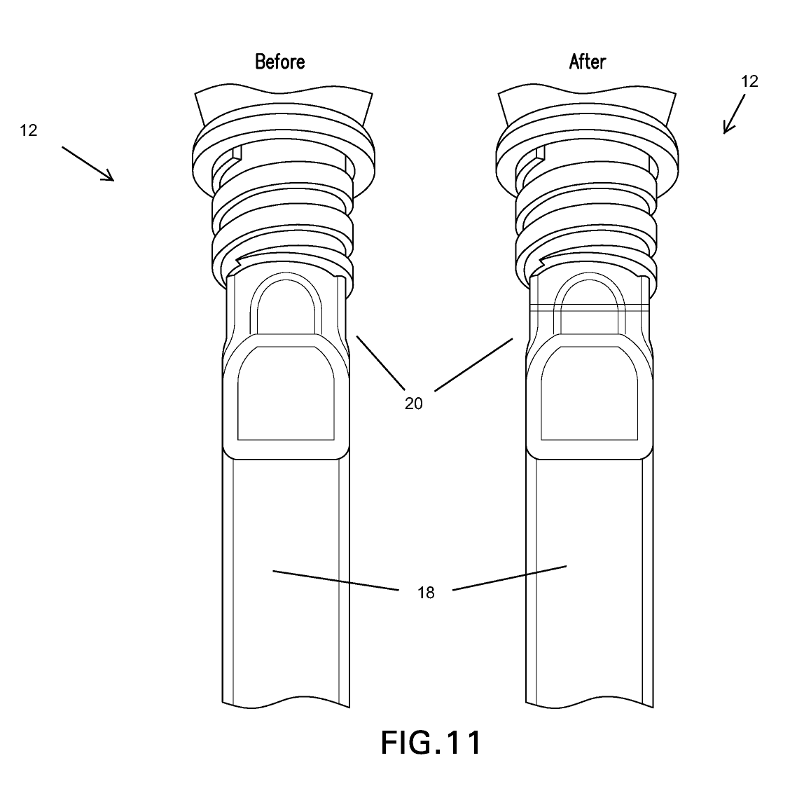

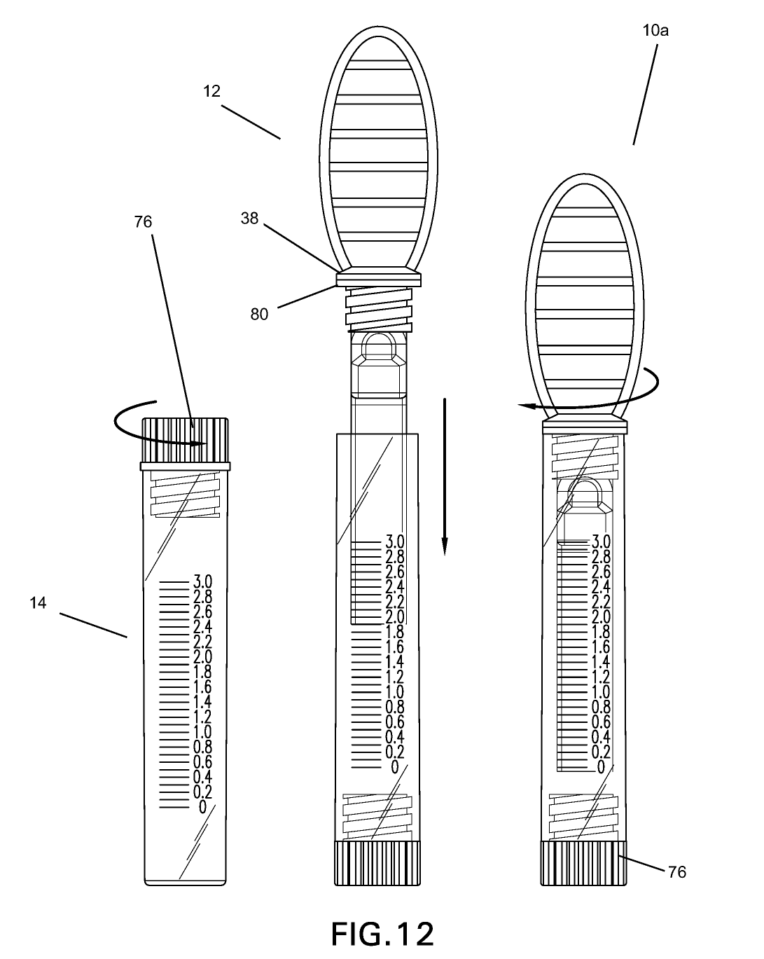

As shown in FIGS. 1-13, an embodiment of a collection system 10 and method for collecting samples is provided. Collection system 10 includes a sample collector 12 and a transport tube 14. Collector 12 includes a handle 16, collector strip 18, and a sample volume adequacy indicator (SVAI) portion 20. Handle 16 extends from a first portion 22 to a second portion 24. Handle first portion 22 includes first and second grip portions 30 and 32 disposed on respective first and second opposing sides 26 and 28. Handle second portion 24 includes a male threaded portion 34 defining an interior cavity 36, and a stop collar 38 adjacent threaded portion 34, which compresses gasket 80 to seal against the open end 70 of transport tube 14.

Collector strip 18 has known absorbency characteristics for a given preselected class of sample fluid, for example human saliva, urine or other fluid. Therefore, for a collector strip 18 made to specified dimensions, full saturation will correspond to a known volume of fluid.

In the embodiment, SVAI portion 20 includes opposed first and second members 40, 42, respectively, each extending from a respective first end 44, 46, to a respective second end 48, 50, and coupled proximate their first ends 44, 46, and defining a slot 52 between first and second members 40, 42, to receive collector strip 18. The outer surfaces 54 and 56 of first and second members 40 and 42, respectively, are shaped to insert snuggly within cavity 36, thereby compressing first and second members 40 and 42 against collector strip 18 when the unit is inserted into cavity 36.

First member 40 is transparent and includes a light pipe, to indicate adequate saturation of the collector strip 18. Light pipe includes a first surface 60, in contact with collector strip 18, a second end surface 62 normal to first surface 60, and a third surface 64 oriented at an angle of incidence to the first surface 60 and also in direct contact with collector strip 18. In the embodiment, second end surface 62 is transparent and the surfaces of interior cavity 36 are a contrasting color from collector strip 18. Alternatively, the interior cavity surfaces could be white or other soft color, and end surface 62 could have a contrasting color applied to it to provide visibility. In the embodiment, second end surface 62 is coextensive with first member first end 44. When collector strip 18 is dry and the collector 12 is viewed directly from the side, the refractive boundary created by the third surface in contact with collector strip 18 will reflect the color at end surface 62, displaying a line or mark. When collector strip 18 becomes saturated to the region in contact with light pipe first and third surfaces 60, 64, the refractive boundary will change such that less light from second end surface 62 is reflected at surface 64, and the line or mark disappears.

In the embodiment, a second SVAI 66 is provided, which may be used in combination with a light pipe or independently, without a light pipe structure. Second SVAI 66 includes a chemical marker applied to collector strip 18 which will visibly change when exposed to saliva, either by changing color, or by changing from nonvisible to visible, or vice versa. In the embodiment SVAI 66 is a chemical marker applied transversely across collector strip 18 which changes color from yellow-green color to blue color when contacted by the sample fluid.

Transport tube 14 extends from a first closed end 68 to a second open end 70. Second end 70 includes first male-threaded portion 72 and a second female-threaded portion 74, the male-threaded portion 72 to receive a sealing transport tube cap 76, and the female-threaded portion 74 to engage the male-threaded portion 34 of handle 12.

Transport tube 14 is transparent or translucent. Transport tube 14 is marked with high precision visually discernable volume indicia 82 at least in the range of normal operating volumes. Transport tube 14 dimensions are selected to be compatible with automated testing equipment. In the embodiment, transport tube 14 outer diameter is 12 mm and length is 75 mm. Transport tube 14 comes pre-loaded with a precise volume of buffer solution 78, sealed by a resealable cap 76 engaged by male-threaded portion 72.

In method and operation, the following steps are provided. The collector 12 may be inserted into a person's mouth, or into a pre-collected fluid sample such as a urine sample, for example, and the SVAI's 58 and 66 are monitored to determine when collector strip 18 is saturated. Collector 12 is then removed from the sample collection area, threaded cap 76 removed from transport tube 14, collector strip 18 inserted into transport tube 14, until collector male-threaded portion 34 engages against transport tube female-threaded portion 74, and the threads are tightened until gasket 80 seals against open end 70. The transport tube/collector assembly (assembled as shown by 10a, FIGS. 1-4) are inverted several times to thoroughly coat collector strip 18 with the buffer solution 78, and is ready for transfer to the testing location. Generally, during transfer to the test location sufficient time will pass (aided by the mechanical agitation from transporting the assembled system 10a) to achieve completely uniform diffusion and mixing of buffer/sample throughout transport tube 14 and saturated collector strip 18.

In preparation for testing, the transport tube/collector 10a is placed upright, collector 12 with collector strip 18 is removed, and a threaded transport tube cap 76 is re-installed to seal transport tube 12. The liquid level is obtained by viewing the visual scale 82, either manually or using an optical measuring device such as a laser scanner or digital camera. The operator can then determine the Corrected Dilution using the Measure Saliva Load. The scale level (SL) indicated by the visual scale after the sample has been thoroughly mixed will be the net result of the collection strip absorbing a portion of the combined buffer solution and saliva sample after immersion and agitation to become fully saturated. When removed, the fully saturated collection strip will remove a known volume of mixed sample with it. With this value known, the actual dilution ratio (sample volume/buffer solution volume) can be precisely determined, so that the test results can be corrected to the actual volume of sample fluid and/or dilution ratio.

Recovery of analytes from the Fluid Collection Device will depend upon the structure of the analyte being tested. Some analytes bind very tightly to cellular material and other components of saliva are trapped by the collection matrix. It is strongly recommended that spiked recovery experiments be performed for each analyte of interest using appropriate protocols. Expect that recovery of sample from the Collection Strip will depend heavily upon time of exposure to the Extraction Buffer and the number of mixing inversions. Transporting samples by mail or long courier times is usually sufficient to ensure maximum extraction/recovery.

Whole saliva dipping recovery measurements may, depending upon reporting requirements, need to be corrected for the amount of saliva absorbed onto the pad. The maximum saliva load by oral (mouth) collection is 1.0-1.1 mL of saliva. This will result in a 1:3 dilution with the 2.0 mL of Extraction Buffer provided at complete extraction (see point 4 below). Dipping into whole saliva usually results in lower recovery than oral [mouth] collection, and typically results in higher dilutions.

The quantity of saliva obtained from any collection can be determined using the graduated scale on the side of the Transport Tube.

(i) Once the loaded Collection Strip has been placed into the Transport Tube and the Handle has been screwed down tightly and inverted 20-40 (twenty to forty) times, allow contents of the Transport Tube to settle. Make sure all fluid is settled below and not trapped in the Handle or the sides of the Transport Tube, by tapping or flicking the Tube.

(ii) Remove the Handle/Collection Strip from the Transport Tube, ensuring that any loose fluid is returned to the tube. Discard the handle/collection strip.

(iii) Turn the Transport Tube so the scale is visible, and level with the naked eye.

(iv) Determine the load volume by reading the scale value (SV) that corresponds to the meniscus level of the Extraction Buffer. Use the lines at 100 μL intervals to judge to within 25-50 μL. The actual Measured Saliva Load (MSL, mL) and the corrected dilution can then be calculated using the equation below:

TABLE 1

| | | Extraction | |
|---|---|---|---|
| Dilution | MSL | Buffer Provided | Corrected |
| Factor | Calculation | (mL) | Dilution |
| 1:3 | SV-0.87 | 2.00 | (MSL + 2.00)/MSL |

An example calculation is provided. If the scale value=1.9, the MSL would equal 1.90−0.87=1.03 mL. At an MSL of 1.03 mL the dilution would be 1.03+2.00/1.03=2.94 or within 2% of a 1:3.

On rare occasions, the SVAI chemical indicator may turn blue with load volumes less than 0.5 mL of sample fluid. Low load volumes may occasionally result in solutions that are too dilute to be measured within the operating parameters of the method. A range of acceptable load values, with or without dilution correct on, can be established within the collection protocols described herein. In rare situations, where it is not possible to establish the correct measured saliva load and/or corrected dilution, it is recommended that the collection process should be repeated with a new device to ensure accurate dilution correction factors.

Absolute recovery of saliva from the Collection Strip will depend upon the method used. Mixing of the sample by inversion after collection alone may not result in complete extraction of the target analyte. Vortexing the Transport Tube with the Collection Strip inside for at least 15 seconds typically results in complete extraction; however the vortexing action can also remove fine particulates from the pad and these particulates may present a problem in some instances when using automated pipetting equipment or other pipetting methods hat use tow bore diameter tips that can clog. In these cases extraction methodology will need to be validated by the laboratory depending upon the final usage. Centrifugation of the tube containing the Collection Strip and Transport Buffer will pellet the fine particulates, and allow unhindered pipetting at the top of the solution column.

Twenty-two samples were tested using artificial saliva over a load range of 0.5 to 1.0 mL. The scales were read by 4 individuals and the scale readings were averaged. The average % error was 1.2% with an average variation of +/0.02 mL (CV), as indicated in the following TABLE 2.

In testing, twenty-one individual samples were loaded until the SVAI changed to a crisp blue line. The average load was 0.82 mL with a range of 0.64-0.92 mL. The average collection time was 68 seconds with a range of sampling times from 22 seconds to 360 seconds. The scales were read by four individuals and the scale readings were averaged. The average % error was 0.6% with an average variation of +/−0.01 mL (CV). These testing results are summarized in the following TABLE 3.

| Donor | Load | Scale | Calculated | % |
|---|---|---|---|---|
| ID | Weight (g) | Reading | Load (ml) | Recovery |
| 1 | 0.824 | 1.70 | 0.83 | 101% |
| 2 | 0.830 | 1.70 | 0.83 | 100% |
| 3 | 0.820 | 0.65 | 0.78 | 95% |
| 4 | 0.914 | 1.81 | 0.94 | 103% |
| 5 | 0.874 | 1.70 | 0.83 | 95% |
| 6 | 0.749 | 1.59 | 0.72 | 96% |
| 7 | 0.919 | 1.73 | 0.85 | 93% |
| 8 | 0.872 | 1.70 | 0.83 | 95% |
| 9 | 0.811 | 1.70 | 0.83 | 102% |
| 10 | 0.640 | 1.50 | 0.63 | 98% |
| 11 | 0.653 | 1.50 | 0.63 | 96% |
| 12 | 0.816 | 1.70 | 0.83 | 102% |
| 13 | 0.872 | 1.80 | 0.93 | 107% |
| 14 | 0.859 | 1.70 | 0.83 | 97% |
| 15 | 0.824 | 1.70 | 0.83 | 101% |
| 16 | 0.830 | 1.70 | 0.83 | 100% |
| 17 | 0.775 | 1.66 | 0.79 | 102% |
| 18 | 0.841 | 1.76 | 0.89 | 106% |
| 19 | 0.851 | 1.71 | 0.84 | 99% |
| 20 | 0.832 | 1.73 | 0.85 | 103% |
| 21 | 0.832 | 1.66 | 0.79 | 95% |
| Average | 0.82 | 1.69 | 0.82 | 99% |
| Std Dev | 0.068 | 0.076 | 0.076 | 0.037 |
| % CV | 8.3% | 4.5% | 9.3% | 3.8% |

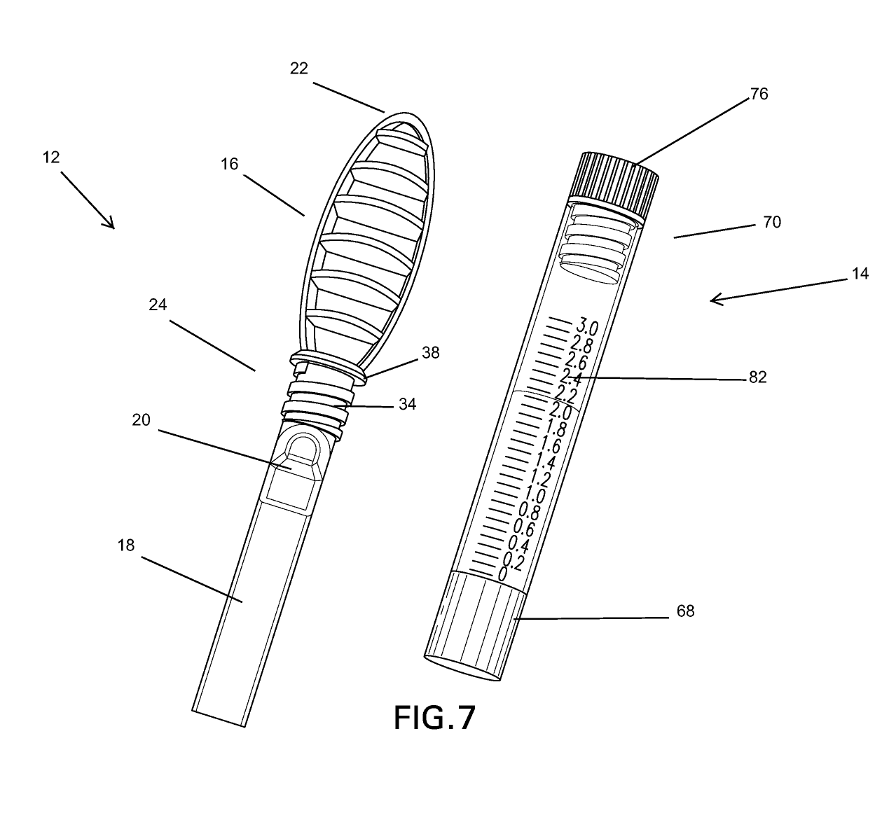

Referring generally to FIGS. 1-6, and particularly to FIGS. 7-14, a method of collecting a fluid sample and providing an accurate dilution factor is described. In the embodiment, the fluid sample is saliva collected directly from a subject's oral cavity:

While holding the Handle, place the Collection Strip in the mouth with the sample volume adequacy indicator visible to the operator. Allow the Collection Strip to absorb saliva that has been pooled and/or secretes during collection. Continue to pool and absorb saliva while moving the Collection Strip further into the mouth with the goal of saturating the entire pad.

Continuously observe the sample volume adequacy indicator and/or remove the Collection Strip from the mouth every 15-20 seconds and continue collecting until the SVAI visibly changes, in this case changing color from yellow/green to blue. Sample collection is now complete.

Remove the cap from the Transport Tube containing the Extraction Buffer and push the cap into the dock located in the bottom of the Transport Tube. Place the saturated pad connected to the Handle into the Transport Tube containing the Extraction Buffer, and screw the cap tightly onto the Transport Tube.

Invert Transport Tube to ensure adequate mixing. Experimental results indicate 20-40 times to be sufficient under most sample collection regimes. The intent is to ensure the buffer solution fully contacts and saturates the collection strip to flush the sample from the collection strip, so that the sample concentration is uniform throughout the bulk solution and collection strip. The buffer solution will then contain an adequate volume of bodily fluid for testing, and will have a known dilution factor to apply a correction to the test results, thereby providing an accurate indication of the target analytes in the original bodily fluid sample.

Place the sealed Transport Tube containing the Collection Strip with handle and Extraction Buffer into an appropriate shipping container.

An operator may then remove the collector and collection strip, re-install the transport tube cap, observe the liquid level and determine the load volume, and the dilution factor, as described above.

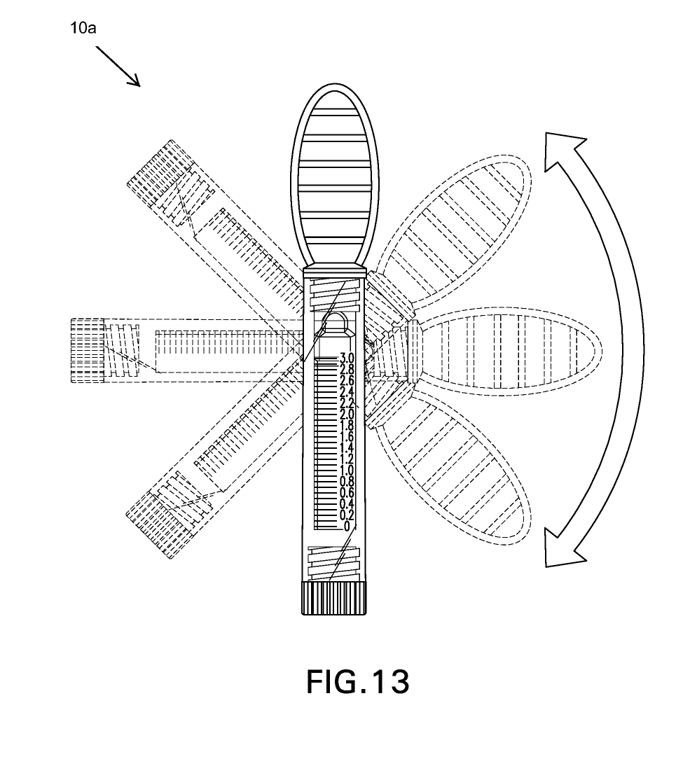

Referring to FIG. 13, the transport tube cap may be left inserted into the transport tube second end coupling to be available for the destination testing facility to use with automated testing and processing equipment, leaving the handle threaded into the transport tube for shipment.

Alternatively, the handle may be removed and the collection strip separated from the handle by removing the SVAI portion 20 from cavity 36, causing slot 52 to decompress and expand slightly and allowing collection strip 18 to slide out. Collection strip 18 can then drop into transport tube 14, and transport tube cap 76 re-installed for transfer to the testing facility.

In addition to the functional elements and methods described and claimed herein, FIGS. 1-4 & 7-8 demonstrate the nonfunctional aesthetic features of the collector design.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be limited by the particular embodiments herein described but should be defined only by the appended claims and equivalents thereof.

We claim:

1. A body fluid sample collection system, comprising:
a sample collector
the sample collector comprising:
a handle the handle extending from a first gripping portion to a second portion, the second portion including a collection strip retainer and male threaded portion disposed around the collection strip retainer,
a collection strip extending out from the collection strip retainer, wherein the collection strip has a pre-determined maximum load for the selected sample fluid; and,
a sample volume adequacy indicator;
a transport tube comprising a cylindrical body extending from a first closed end to a second open end, the first end further including a coupling to removably receive a transport tube cap, the second end further including a male threaded portion to removably engage a transport tube cap and a female threaded portion to removably receive the sample collector male threaded portion, the transport tube cylindrical body further including visually discernable graduated scale and wherein the transport tube cylindrical body is either translucent or transparent;
a transport tube cap having a threaded portion to removably gage the transport tube second end; and,
a predetermined volume of buffer solution disposed within the transport tube cylinder.

2. The system of claim 1, further comprising:
the collection strip removably coupled to the collection strip retainer.

3. The system of claim 2, further comprising:
the sample volume adequacy indicator formed into the collection strip retainer.

4. A method for collecting a target body fluid sample having a known dilution factor, comprising the steps of:
providing a sample collector and a resealable transport tube having a predetermined volume of buffer solution therein and a visually discernable graduated scale, the sample collector including a collection strip having a predetermined maximum load for the target bodily fluid, the sample collector further including a sample volume adequacy indicator;
inserting at least a portion of the collection strip into a target body fluid sample point;
removing the sample collector from the collection location upon the occurrence of either the sample volume adequacy indicator indicating sufficient volume or a predetermined maximum sample collection time;
insert the sample collector into the transport tube such that the collection strip is in contact with the buffer solution;
agitating the transport tube with the inserted collector by at least causing the buffer solution to wash over the length of the collection strip a plurality of times;
removing the collector and collection strip;
observing the graduated scale value; and,
using the graduated scale value, determining the dilution correction.
